(19) United States
(12) Patent Application Publication
(10) Pub. No.: US 2024/0216435 A1
(43) Pub. Date: Jul. 4, 2024

(54) COMPOSITION FOR SKIN REGENERATION, WHITENING, ANTIOXIDATION, OR WOUND TREATMENT, COMPRISING STEM CELL-DERIVED EXOSOME AS ACTIVE INGREDIENT, AND USE THEREOF

(71) Applicant: EHLBIO Co., Ltd., Uiwang-si (KR)

(72) Inventors: Hong-Ki LEE, Seoul (KR); Seong-Hun LEE, Anyang-si (KR); Sang-Heon KIM, Incheon (KR); Jeong-Ah JIN, Seoul (KR); Tae-Wook KANG, Seoul (KR); Su-Bin SO, Incheon (KR); Jin-Sang YOON, Seoul (KR); Young-Jun LEE, Seoul (KR); Hyung-Joon SO, Anyang-si (KR)

(21) Appl. No.: 18/546,831

(22) PCT Filed: Feb. 7, 2022
(86) PCT No.: PCT/KR2022/001851

§ 371 Date: Mar. 14, 2024

(30) Foreign Application Priority Data
Feb. 17, 2021 (KR) 10-2021-0021026

(51) Int. Cl. A61K 35/22; A61K 8/98; A61P 17/02; A61Q 19/00; A61Q 19/02; C12N 5/074

(52) U.S. Cl. CPC A61K 35/22; A61K 8/981; A61P 17/02; A61Q 19/00; A61Q 19/02; C12N 5/0607

(57) ABSTRACT

The present invention relates to a composition comprising, as an active ingredient, a stem cell-derived exosome having an improved skin regeneration, whitening, or antioxidation function. More specifically, the present invention relates to a cosmetic composition or pharmaceutical composition for skin regeneration, whitening, antioxidation, or wound treatment, comprising urine-derived stem cell-derived exosome as an active ingredient.

### TECHNICAL FIELD

The present disclosure relates to a composition including exosomes derived from stem cells as an active ingredient with enhanced skin regeneration, skin whitening, or antioxidant functions. More specifically, the present disclosure relates to a cosmetic composition or pharmaceutical composition including urine stem cell-derived exosomes as an active ingredient for skin regeneration, whitening, antioxidant, or wound healing.

### BACKGROUND ART

Urine-derived stem cells represent a new source of stem cells that can be obtained through a simple separation process from human urine. These cells offer the advantage of being able to be obtained multiple times without causing any pain or after-effects to the patient, compared to other mesenchymal stem cells. As a result, a larger number of stem cells necessary for initial culture can be obtained compared to traditional cell sources. Additionally, urine-derived stem cells a high proliferation rate, allowing a large number of cells to be obtained in a short period of time and thus increasing their potential for commercialization. The low expression of HLA-DR means they do not induce an immune response when transplanted in the body, making them highly feasible for clinical as autologous application cell therapy. Furthermore, urine-derived stem cells are recognized for their therapeutic potential, such as secreting various cytokines, including growth factors, in appropriate culture environments.

Recent studies report that biological factors that regulate cell activity are included in secretomes, which are secretions from cells. Exosomes are nano-vesicles ranging from 30 to 150 nm in size that are secreted from cells[1,2]. Known to contain genetic material such as mRNA and miRNA, as well as protein transport substances like cytokines, exosomes are specific to the cell type and regulated differently depending on the secretion environment. Exosomes serve as signaling mediators between cells, regulating various cell activities such as activation, growth, migration, differentiation, de-differentiation, apoptosis, and necrosis. They also contain specific genetic material and bioactive factors according to the nature and state of the originating cell. In particular, stem cell-derived exosomes contain genes, proteins, and growth factors related to stem cell proliferation, differentiation, and regeneration, playing an important role in tissue regeneration[3,4,5].

With the increase in the aging population and the development of various skin beauty technologies, consumers' desire to improve skin aging is growing. Active research on stem cell exosomes, and the development of functional exosome cosmetics that are safe and highly effective, is a major interest in the cosmetics industry. Stem cell exosomes can be used as anti-aging cosmetic ingredients that can improve skin aging phenomena, and active research is being conducted on this topic.

Therefore, the present inventors sought to efficiently separate and purify exosomes secreted during the proliferation of urine-derived stem cells, verify the efficacy of urine-derived stem cell exosomes in wound healing, wrinkle reduction, and skin whitening, and develop a new functional cosmetic with no side effects and excellent efficacy on the human body.

### RELATED ART DOCUMENTS

[Non-patent literature 1] S, E. L. A., Mager, I., Breakefield, X. O. & Wood, M. J. Extracellular vesicles: biology and emerging therapeutic opportunities. Nature reviews. Drug discovery 2013. 12, 347-357.
[Non-patent literature 2] Sahoo, S. et al. Exosomes from human CD34 (+) stem cells mediate their proangiogenic paracrine activity. Circulation research, 2011. 109, 724-728.
[Non-patent: literature 3] Schorey, J. S. & Bhatnagar, S. Exosome function: from tumor immunology to pathogen biology. Traffic, 2008, 9, 871-881.
[Non-patent literature 4] Thery, C., Zitvogel, L. & Amigorena, S. Exosomes: composition, biogenesis and function. Nature reviews. Immunology, 2002. 2, 569-579.
[Non-patent literature 5] Vishnubhatla, I., Corteling, R., Stevanato, L., Hicks, C. & Sinden, J. The Development of Stem Cell-derived Exosomes as a Cell-free Regenerative
Medicine. Journal of Circulating Biomarkers, 2014. 1.

### DISCLOSURE OF INVENTION

### Technical Problem

The present inventors have previously disclosed a medium composition for culturing urine-derived stem cells and a method for culturing urine-derived stem cells using the same in Korean Patent Application No. 10-2019-0139702. In the course of continuously conducting research on the urine-derived stem cells, the present inventors have confirmed that exosomes secreted from urine-derived stem cells have an efficacy in improving skin aging, such as wound healing, wrinkle reduction, and skin whitening, leading to the present disclosure.

### Solution to Problem

An aspect of the present disclosure is to provide a cosmetic composition including exosomes derived from urine-derived stem cells as an active ingredient for skin regeneration, skin whitening, antioxidation, or wound healing.

Another aspect of the present disclosure is to provide a pharmaceutical composition including exosomes derived from urine-derived stem cells as an active ingredient for skin regeneration, skin whitening, antioxidation, or wound healing.

Another of the present disclosure is to provide a food composition including exosomes derived from urine-derived stem cells as an active ingredient for skin regeneration, skin whitening, antioxidation, or wound healing.

Another of the present disclosure is to provide a method for producing exosomes derived from urine-derived stem cells for use in skin regeneration, skin whitening, antioxidation, or wound healing.

The term “stem cells”, as used herein, refers to cells that have the potential to differentiate into cells of all tissues in an organism, possessing pluripotency or totipotency, and also exhibit self-renewal capability. The term is intended to encompass embryonic stem cells, induced pluripotent stem cells, and adult stem cells.

As used herein, the term “proliferation” of stem cells refers to the process where the stem cells divide, maintaining their stem cell characteristics without differentiating into specific cells, and thus increasing the total number of cells.

As used herein, the term “culture medium” refers to a composition that contains nutrients necessary for maintaining the growth and survival of cells in vitro.

The term “cell therapy product”, as used herein, refers to cells and tissues that have been isolated, cultured, and prepared from a subject through special manipulation and are used as pharmaceuticals for the purpose of treatment, diagnosis, and prevention. It means a pharmaceutical for use in treating, diagnosing, and preventing diseases through a series of actions including proliferating and selecting living autologous cells, allogeneic cells, or xenogeneic cells in vitro or changing the biological characteristics of cells in other ways for the purpose of restoring the function of cells or tissues.

As used herein, the term “exosome” refers to a nanovesicle of 30-150 nm in size that is secreted from cells and includes genetic material such as mRNA, miRNA, and protein carriers like cytokines.

### Advantageous Effects of Invention

The composition containing exosomes derived from urine-derived stem cells according to the present disclosure is safe with no cytotoxicity (Example 3), has an excellent cell proliferation rate (Example 4), and exhibits the effects of wound healing (Example 5), skin wrinkle reduction, (Example 6), and skin whitening (Example 8). Therefore, the composition containing exosomes derived from urine-derived stem cells according to the present disclosure is expected to be used as a cosmetic composition, a pharmaceutical composition, and a food composition for skin regeneration, skin whitening, anti-oxidation, or wound healing.

### BRIEF DESCRIPTION OF DRAWINGS

FIG. 1 is a plot of protein levels in exosomes isolated by the TFF method of Example 1.

FIG. 2 shows particle size distributions of exosomes isolated according to Example 1.

FIG. 3 shows markers found in exosomes isolated according to Example 1.

FIG. 4 shows plots of cytotoxicity exosomes isolated according to Example 1.

FIG. 5 shows cell proliferation rates of exosomes isolated according to Example 1.

FIG. 6 shows the effect of exosomes isolated according to Example 1 on the migration of human skin fibroblasts and keratinocytes.

FIG. 7 shows the wrinkle reduction effect of exosomes isolated according to Example 1.

FIG. 8 shows the skin whitening effect of exosomes isolated according to Example 1.

### BEST MODE FOR CARRYING OUT THE INVENTION

According to the first embodiment,
the present disclosure aims to provide a cosmetic composition including exosomes derived from urine-derived stem cells as an active ingredient for skin regeneration, skin whitening, antioxidation, or wound healing.

In the cosmetic composition according to the present disclosure, the exosomes can be separated and purified during the culture process of the urine-derived stem cells, and the culture medium composition for culturing the urine-derived stem cells is as described in the Korean Patent Application No. 10-2019-0139702 issued to the present inventors. For example, the culture medium composition for culturing the urine-derived stem cells includes a basic medium containing Dulbecco's Modified Eagle Medium (DMEM) and Ham's F12 at a 3:1 ratio, 2.5% or less of fetal bovine serum, and an additive, wherein the additive is at least one selected from a group consisting of bovine serum albumin, a lipid mixture (chemically defined lipids), FGF23 (fibroblast growth factor 23), T3 (triiodothyronine), and calcifediol. Preferably, the culture medium composition for culturing the urine-derived stem cells includes or consists of a basic medium containing Dulbecco's Modified Eagle Medium (DMEM) and Ham's F12 at a 3:1 ratio, 2.5% or less of fetal bovine serum, bovine serum albumin, a lipid mixture (chemically defined lipids), FGF23 (fibroblast growth factor 23), T3 (triiodothyronine), and calcifediol. The medium composition for extracting exosomes from the urine-derived stem cells may be serum-free and antibiotic-free. The serum-free and antibiotic-free medium composition includes a basic medium containing or composed of Dulbecco's Modified Eagle Medium (DMEM) and Ham's F12 at a 3:1 ratio, fibroblast growth factor (FGF2), hydrocortisone, T3 (triiodothyronine), calcitriol, insulin-transferrin-selenium-ethanolamine, L-ascorbic acid 2-phosphate, and retinoic acid.

In the cosmetic composition according to the present disclosure, the urine-derived stem cells may be autologous, allogeneic, or xenogeneic.

In the cosmetic composition according to the present disclosure, the wound may include cuts, incisions (e.g., surgical incisions), abrasions, thermal injuries or lacerations, fractures, contusions, burns, or amputations.

The cosmetic composition according to the present disclosure may include: ingredients commonly used in cosmetic compositions, other than the active ingredient, for example, fatty substances, organic solvents, solvents, thickeners and gelling agents, softeners, antioxidants, suspending agents, stabilizers, foaming agents, fragrances, surfactants, water, ionic or nonionic emulsifiers, fillers, metal ion sequestrants and chelating agents, preservatives, vitamins, sunscreens, humectants, essential oils, dyes, pigments, hydrophilic or lipophilic active agents, conventional adjuvants such as lipid vesicles, and excipients.

According to the present disclosure, the cosmetic composition may be manufactured in any formulation typically produced in the related industry. For example, it may be formulated into a solution, suspension, emulsion, paste, gel, cream, lotion, powder, oil, powder foundation, emulsion foundation, wax foundation, spray, etc., but is not limited thereto. More specifically, it may be manufactured into cosmetic formulations such as skin softeners, toners, astringents, lotions, milk lotions, moisturizing lotions, nutritional lotions, massage creams, nutritional creams, eye creams, moisturizing creams, hand creams, essences, nutritional essences, packs, cleansing foams, cleansing waters, cleansing lotions, cleansing creams, body lotions, body cleansers, soaps, and powders.

If the formulation of the cosmetic composition is a paste, cream, or gel, it can use animal fats, plant oils, waxes, paraffins, starch, tragacanth, cellulose derivatives, polyethylene glycol, silicones, bentonite, silica, talc, or zinc oxide as a carrier.

If the formulation of the cosmetic composition is a powder or spray, it can use lactose, talc, silica, aluminum hydroxide, calcium silicate, or polyamide powder as a carrier, and particularly for sprays, it can include propellants such as chlorofluorocarbons, propane/butane, or dimethyl ether.

For the cosmetic composition as a solution or emulsion, solvents, solubilizers, or emulsifiers, such as water, ethanol, isopropanol, ethyl carbonate, ethyl acetate, benzyl alcohol, benzyl benzoate, propylene glycol, 1,3-butylene glycol, glycerol esters, polyethylene glycol, or sorbitan fatty acid esters, may be used as carrier ingredients.

If the formulation of the cosmetic composition is a suspension, it can use, as carriers, diluents such as water, ethanol, or propylene glycol, suspending agents such as ethoxylated isostearyl alcohol, polyoxyethylene sorbitol ester, and polyoxyethylene sorbitan ester, and thickeners such as microcrystalline cellulose, aluminum hydroxide, bentonite, agar, or tragacanth.

According to the second embodiment,
the present disclosure also aims to provide a pharmaceutical composition including exosomes derived from urine-derived stem cells as an active ingredient for skin regeneration, whitening, antioxidation, or wound healing.

In the pharmaceutical composition according to the present disclosure, the exosomes can be separated and purified during the cultivation process of the urine-derived stem cells, and the culture medium composition for cultivating the urine-derived stem cells is as disclosed in Korean Patent Application No. 10-2019-0139702 issued to the present inventors. For example, the culture medium composition for cultivating the urine-derived stem cells includes a basic medium containing Dulbecco's Modified Eagle Medium (DMEM) and Ham's F12 at a ratio of 3:1, 2.5% or less fetal bovine serum, and an additive wherein the additive is at least one selected from a group consisting of bovine serum albumin, chemically defined lipids, FGF23 (fibroblast growth factor 23), T3 (triiodothyronine), and calcifediol. Preferably, the culture medium composition for cultivating the urine-derived stem cells includes or is composed of a basic medium containing Dulbecco's Modified Eagle Medium (DMEM) and Ham's F12 at a ratio of 3:1, less than 2.5% fetal bovine serum, bovine serum albumin, chemically defined lipids, FGF23 (fibroblast growth factor 23), T3 (triiodothyronine), and calcifediol.

In the pharmaceutical composition according to the present disclosure, the urine-derived stem cells may be autologous, allogeneic, or xenogeneic.

In the pharmaceutical composition according to the present disclosure, the wounds may include cuts, incisions (for example, surgical incisions), abrasions, thermal injuries or lacerations, fractures, contusions, burns, or amputations.

The pharmaceutical composition according to the present disclosure may include a pharmaceutically acceptable diluent or carrier. The diluent may be lactose, corn starch, soybean oil, amorphous cellulose, or mannitol, and the lubricant may be magnesium stearate, talc, or a combination thereof. The carrier may be a bulking agent, a disintegrant, a binder, a lubricant, or a combination thereof. The bulking agent may be microcrystalline cellulose, lactose, low-substituted hydroxypropyl cellulose, or a combination thereof. The disintegrant may be calcium carboxymethylcellulose, sodium starch glycolate, anhydrous dicalcium phosphate, or a combination thereof. The binder may be polyvinylpyrrolidone, low-substituted hydroxypropylcellulose, hydroxypropylcellulose, or a combination thereof. The lubricant may be magnesium stearate, silicon dioxide, talc, or a combination thereof.

The pharmaceutical composition may be formulated into a non-oral administration formulation. Non-oral administration formulations may include injections or topical skin formulations. Topical skin formulations may be creams, gels, ointments, skin emulsions, skin suspensions, transdermal patches, mask packs, medicated bandages, lotions, or a combination thereof. The topical skin preparations can be appropriately blended, if necessary, with ingredients typically used in cosmetics or topical pharmaceuticals, for example, aqueous ingredients, oil-based ingredients, powder ingredients, alcohols, humectants, thickeners, UV absorbers, whitening agents, preservatives, antioxidants, surfactants, fragrances, dyes, various skin nutrients, or a combination thereof. The topical skin preparations may also be appropriately formulated with metal blockers such as disodium edetate, trisodium edetate, sodium citrate, sodium polyphosphate, sodium metaphosphate, and gluconic acid, drugs such as caffeine, tannin, verapamil, licorice extract, glabridin, hot water extract of kaolin fruit, various raw medicines, acetate tocopherol, glycyrrhizic acid, tranexamic acid and its derivatives or salts thereof, vitamin C, magnesium ascorbyl phosphate, ascorbyl glucoside, arbutin, kojic acid, glucose, fructose, trehalose and other sugars.

According to the third embodiment,
the present disclosure also aims to provide a food composition including exosomes derived from urine-derived stem cells as an active ingredient for skin regeneration, skin whitening, antioxidation, or the prevention or relief of wounds.

In the food composition according to the present disclosure, the exosomes can be separated and purified in the process of culturing the urine-derived stem cells, and the medium composition for culturing the urine-derived stem cells is as described in Korean Patent Application No. 10-2019-0139702 filed by the inventors of the present disclosure. For example, the culture medium composition for culturing the urine-derived stem cells includes a basic medium containing Dulbecco's Modified Eagle Medium (DMEM) and Ham's F12 at a 3:1 ratio, 2.5% or less of fetal bovine serum, and an additive, wherein the additive is at least one selected from a group consisting of bovine serum albumin, a lipid mixture (chemically defined lipids), FGF23 (fibroblast factor 23), T3 growth (triiodothyronine), and calcifediol. Preferably, the culture medium composition for culturing the urine-derived stem cells includes or consists of a basic medium containing Dulbecco's Modified Eagle Medium (DMEM) and Ham's F12 at a 3:1 ratio, 2.5% or less of fetal bovine serum, bovine serum albumin, a lipid mixture (chemically defined lipids), FGF23 (fibroblast growth factor 23), T3 (triiodothyronine), and calcifediol.

In the food composition according to the present disclosure, the urine-derived stem cells may be autologous, allogeneic, or xenogeneic.

In the food composition according to the present disclosure, the wounds include cuts, incisions (e.g., surgical incisions), abrasions, thermal wounds or lacerations, fractures, contusions, burns, or amputations.

The food composition according to the present disclosure can additionally include sweeteners, flavorings, physiologically active ingredients, minerals, etc. in addition to the active ingredients.

The food composition according to the present disclosure may include preservatives, emulsifiers, acidifiers, thickeners, etc. as needed. These preservatives, emulsifiers, and the like are used in trace amounts so long as the purpose of their addition is achieved. As used numerically expressed, the trace amount means a range of about 0.0005% by weight to about 0.5% by weight, based on the total weight of the food composition.

According to a fourth embodiment,
the present disclosure aims to provide a method for producing exosomes derived from urine-derived stem cells for use in skin regeneration, whitening, antioxidant, or wound healing.

This method includes the steps of: culturing urine-derived stem cells in a medium composition including a basic medium containing Dulbecco's Modified Eagle Medium (DMEM) and Ham's F-12 at a 3:1 ratio, 2.5% or less of fetal bovine serum, and an additive, and
culturing the cultured urine-derived stem cells in a serum-free and antibiotic-free medium composition to extract exosomes.

In the method according to the present disclosure, the additive includes or is composed of bovine serum albumin, a lipid mixture (chemically defined lipids), FGF23 (fibroblast growth factor 23), T3 (triiodothyronine), and calcifediol.

In the method according to the present disclosure, the serum-free and antibiotic-free culture solution includes or is composed of a basic medium containing Dulbecco's Modified Eagle Medium (DMEM) and Ham's F-12 at a 3:1 ratio, fibroblast growth factor 2 (FGF2), hydrocortisone, T3 (triiodothyronine), calcitriol, insulin-transferrin-selenium-ethanolamine, L-ascorbic acid 2-phosphate, and retinoic acid.

A better understanding of the present disclosure may be obtained via the following Examples and Test Examples which are set forth to illustrate, but are not to be construed to limit the present disclosure.

### EXAMPLES

### Example 1. Extraction of Exosomes from Human Urine-Derived Stem Cells

Human urine-derived stem cells were cultured up to passages 2 to 7, and then the medium was switched to a serum-free and antibiotic-free medium for the extraction of exosomes. Specifically, human urine-derived stem cells were cultured up to passage 2 to 7 using the medium composition for the culture of urine-derived stem cells disclosed in Korean Patent Application No. 10-2019-0139/02 issued to the present inventors (see Tables 1 and 2 below), and then, the medium was replaced with a serum-free and antibiotic-free medium (see Table 3 below) for the extraction of exosomes, followed by culturing for 24 to 96 hours.

TABLE 1

| Basal media | Supplement | Concentration | |
|---|---|---|---|
| DMEM (Low | FBS | 2.5% | |
| glucose) + | Bovine serum albumin | 0.1-50 | mg/ml |
| Ham's F12 | Chemically defined lipids | See Table 2 | |
| (3:1 mix) | FGF23 | 1-500 | ng/ml |
| | Triiodothyronine (T3) | 0.1-100 | nM |
| | Calcifediol | 10-1000 | nM |

TABLE 2

| Chemically defined lipids | Concentration | |
|---|---|---|
| Arachidonic acid | 2-20 | ng/ml |
| Cholesterol | 220-2200 | ng/ml |
| DL-alpha-Tocopherol Acetate | 70-700 | ng/ml |
| Linoleic acid | 10-100 | ng/ml |
| Myristic acid | 10-100 | ng/ml |
| Oleic acid | 10-100 | ng/ml |
| Palmitic acid | 10-100 | ng/ml |
| Palmitoleic acid | 10-100 | ng/ml |
| Pluronic F-68 | 90-900 | μg/ml |
| Stearic acid | 10-100 | ng/ml |
| Tween 80 | 2.2-22 | μg/ml |

TABLE 3

| Basal media | Supplement | Concentration | |
|---|---|---|---|
| DMEM (Low | EGF | 0.1-20 | ng/ml |
| glucose) + | FGF2 | 1-500 | ng/ml |
| Ham's F12 | Hydrocortisol | 1-300 | ng/ml |
| (3:1 mix) | Triiodothyronine (T3) | 0.1-100 | nM |
| | Calcitriol | 10-1000 | nM |
| | Insulin-Transferrin-Selenium- | 0.1-10% | |
| | Ethanolamine | | |
| | L-Ascorbic acid 2-phosphate | 1-1000 | nM |
| | Retinoic acid | 0.1-100 | μM |

Afterwards, the cell culture supernatant was collected and centrifuged at 300×g to 500×g for 5 to 15 minutes to remove the cell pellet. Cell debris was removed by high-speed centrifugation at 12000×g to 20000×g for to 60 minutes. Subsequently, the cell culture medium thus free of cells and cell debris was filtered through a 0.22-μm filter or through a TFF (Tangential Flow Filtration) filter with a MWCO (Molecular Weight Cut Off) of 500 kDa to 1000 kDa to remove particles larger than 100 nm to 300 nm. For the separation of exosomes, the filtrate was concentrated using a TFF filter with a MWCO of 30 kDa to 100 kDa, thereby removing particles smaller than 10 nm to 30 nm. The above filtration step was performed until the volume of the concentrate was reduced to 1/50 to 1/100 of its original volume. The concentrated exosomes were washed with phosphate-buffered saline (PBS). At this time, the PBS was added in a volume of 10 to 20 times the volume of the concentrate and concentrated using a TFF filter. The washing step was performed 1 to 10 times. A cosmetic composition was prepared using the exosomes separated and purified through the above process, and used in the following experiments. To compare the efficacy of exosomes derived from human urine-derived stem cells, exosomes were extracted from human adipose-derived stem cells in the same manner as a control group.

### Example 2. Characterization of Isolated Exosomes (BCA Quantification, NTA, Exo-Check)

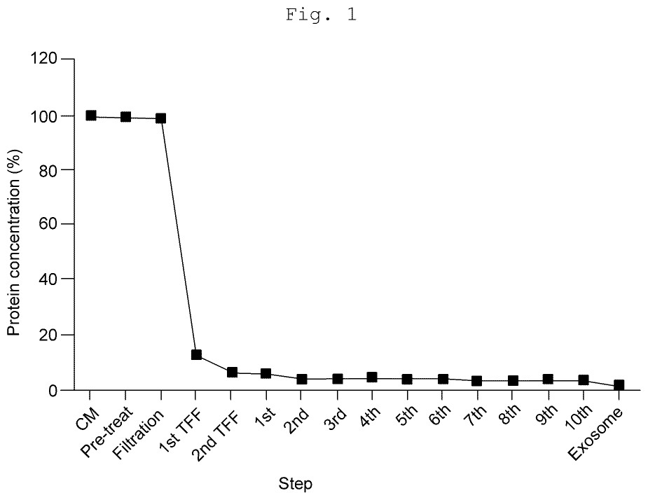

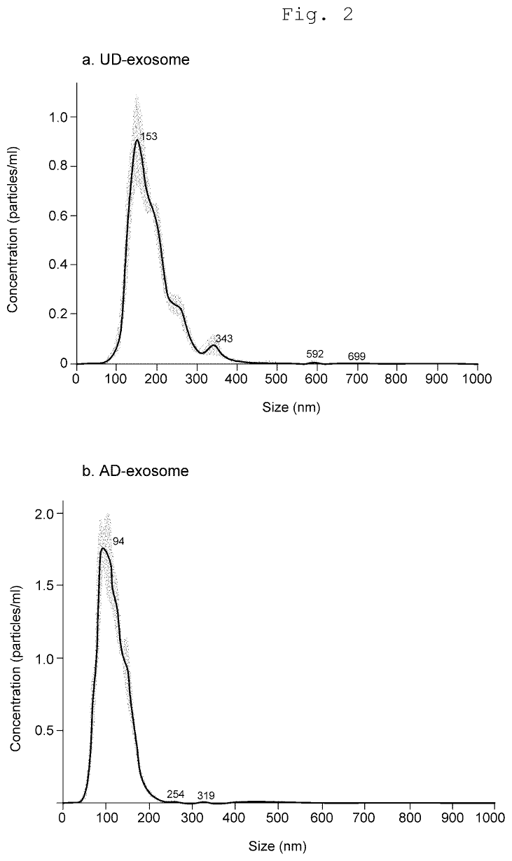

The protein amounts of the exosomes extracted from human urine-derived stem cells (UD-exo) according to Example 1 and exosomes extracted from adipose-derived stem cells (AD-exo) as a control group was measured using the BCA colorimetric method (ThermoFisher Scientific). The degree of separation, concentration, and removal of proteins, lipids, nucleic acids, low molecular weight compounds, etc., by the TFF method was measured by a protein quantification method. As a result, it was shown that the protein present in the culture medium was very effectively removed by the TFF method according to Example 1 (FIG. 1).

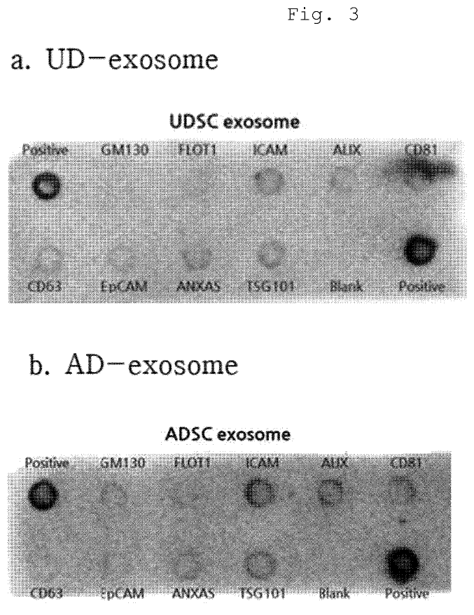

The particle size of the exosomes separated according to the method of Example 1 was measured five times repeatedly using nanoparticle tracking analysis (NTA). As a result, it was shown that size distribution of the separated exosomes was uniform (FIG. 2).

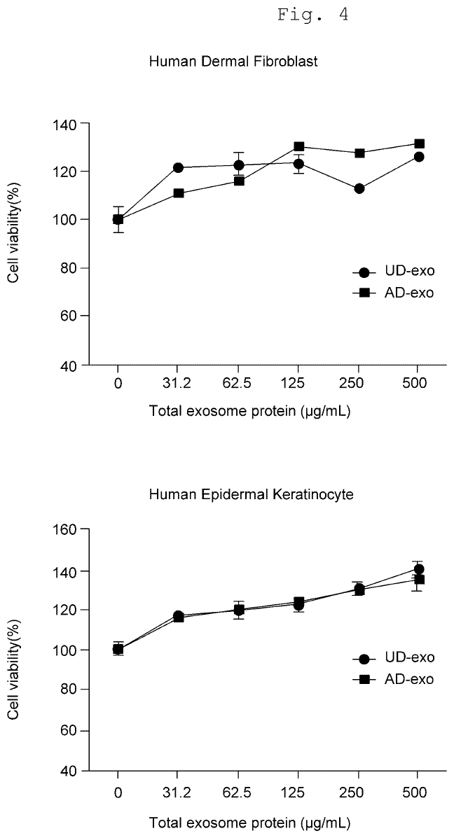

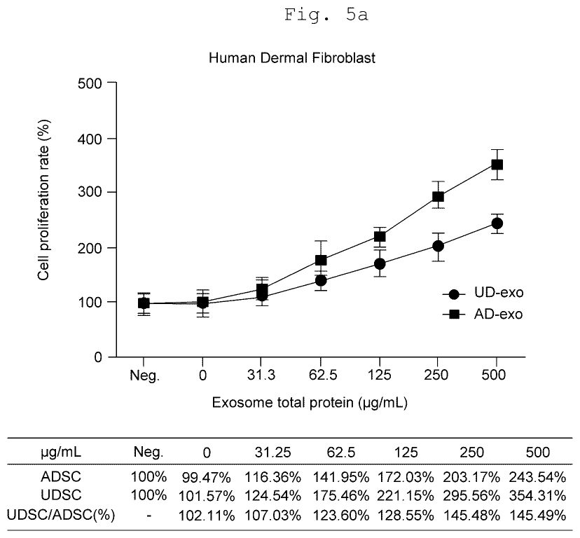

Also, the exosome markers in the exosomes separated by the method according to Example 1 were analyzed using the Exo-check kit (system bioscience). As a result, it was shown that the markers CD63, CD81, and EpCAM were present in the separated exosomes (FIG. 3).

### Example 3. Cytotoxicity According to Exosome Treatment

To evaluate the toxicity of the exosomes obtained by the separation method of Example 1, exosomes were applied at various concentrations to human skin fibroblasts and human keratinocytes and measured for cytotoxicity. Human skin fibroblasts human and keratinocytes were suspended in DMEM supplemented with 10% FBS, seeded at a density of 80 to 90% and cultured for 24 hours at 37° C. in a 5% CO₂ incubator. After 24 hours, the culture medium was replaced with DMEM without FBS, and exosomes extracted from human urine-derived stem cells prepared from Example 1 and exosomes extracted from human adipose-derived stem cells as a control group were applied at predetermined concentrations to the cells. During incubation for 24 to 48 hours, cell viability was evaluated. Cell viability was measured using CCK-8 (Cell Counting Kit-8, cat #CK04-11), MTT (abcam, cat #ab211091), and a microplate reader.

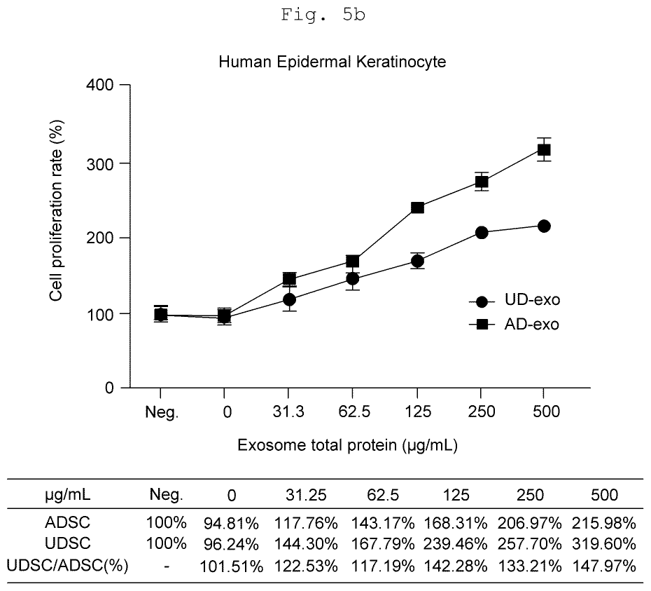

As a result, based on the cell group cultured for the same time in DMEM not treated with exosomes, it was confirmed that there was no cytotoxicity caused by exosomes extracted from human urine-derived stem cells and exosomes extracted from human adipose-derived stem cells within the tested concentration range (FIG. 4).

### Example 4. Cell Proliferation Rate According to Exosome Treatment

In order to evaluate the proliferation rate of human skin fibroblasts and human keratinocytes according to the treatment of the exosomes obtained by the separation method of Example 1, the exosomes were applied at various concentrations to the human skin fibroblasts and human keratinocytes and measured for cell proliferation rate. Human skin n fibroblasts and human keratinocytes were suspended in DMEM supplemented with 10% FBS, seeded at a density of 80 to 90%, and cultured for 24 hours at 37° C. in a 5% CO₂ incubator. After 24 hours, the culture medium was replaced with DMEM without FBS, and exosomes extracted from human urine-derived stem cells prepared from Example 1 and exosomes extracted from human adipose-derived stem cells as a control group were applied at predetermined concentrations to the cells. During incubation for 24 to 48 hours, cell proliferate rates were evaluated. Cell proliferation rates were measured using CCK-8 (Cell Counting Kit-8, cat #CK04-11), MTT (abcam, cat #ab211091), and a microplate reader.

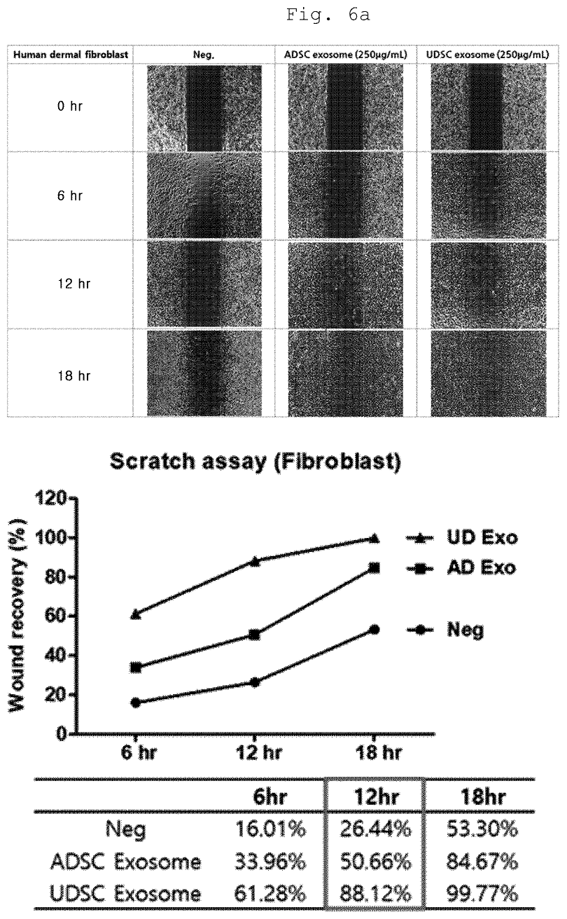

As a result, based on the cell group cultured for the same time in DMEM without exosome treatment, it was shown that the exosomes extracted from human urine-derived stem cells had better cell proliferation rates over the entire concentration of the tested exosomes than the exosomes extracted from human adipose-derived stem cells (FIG. 5).

### Example 5. Effects of Exosomes Extracted from Human Urine-Derived Stem Cells on Migration of Human Skin Fibroblasts and Human Keratinocytes

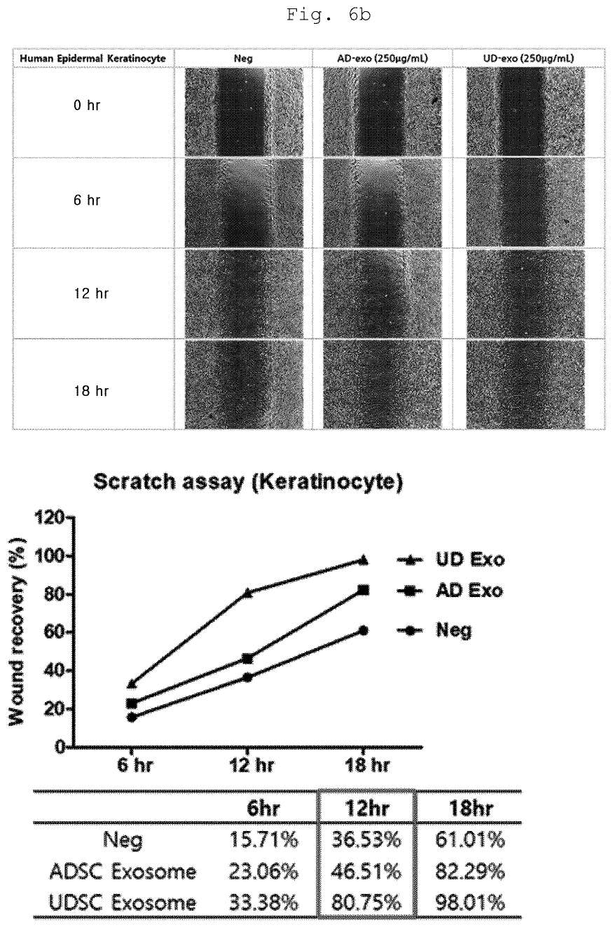

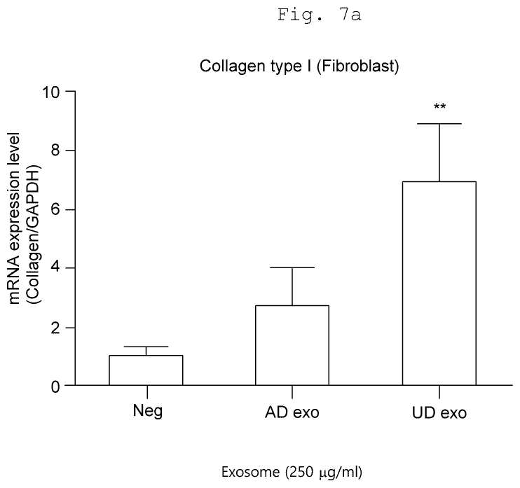

To investigate the effect of exosomes extracted from human urine-derived stem cells (UD-exo) on the migration of human skin fibroblasts and human keratinocytes, use was made of medium compositions containing exosomes extracted from the culture medium of human urine-derived stem cells and exosomes extracted from human adipose-derived stem cells (AD-exo), respectively. The medium compositions were prepared by adding UD-exo at a concentration of 250 μg/mL to DMEM serum-free culture medium and AD-exo at a concentration of 250 μg/mL to DMEM serum-free culture medium, respectively. DMEM serum-free medium was used as a negative control. Human skin fibroblasts and human keratinocytes were each seeded at a density of 2×10⁵ cells/well in a scratch assay mold installed in a 24-well plate, respectively, and incubated in culture medium (DMEM supplemented with 10% fetal bovine serum and 1% penicillin/streptomycin) for 24 hours. After incubation, the installed mold was carefully withdrawn to create wounds at regular intervals, and the medium composition containing exosomes was applied to each cell. After treating the cells with the medium containing exosomes, photographs were taken at 0, 6, 12, and 18 hours, and the extent of cell migration was quantified through Image J. From the 12-hour mark, a higher degree of migration was observed in the cells treated with the medium containing UD-exo (88.12%) than the negative control (26.44%) and the cells treated with AD-exo (50.66%). After 18 hours, the medium containing UD-exo allowed the fibroblasts to migrate faster than the medium containing AD-exo, indicating a superb migration effect on fibroblasts in the UD-exo than AD-exo. As for the keratinocytes, a higher degree of migration was also observed in the cells treated with the medium containing UD-exo (80.75%) from the 12-hour mark than the negative control (36.53%) and the cells treated with AD-exo (46.51%) (FIG. 6). Thus, it has been proven that exosomes extracted from human urine-derived stem cells have a better effect on wound healing than exosomes extracted from human adipose-derived stem cells.

### Example 6. Anti-Wrinkle Effect of Exosomes Extracted from Human Urine-Derived Stem Cells

In order to investigate the effect of exosomes extracted from human urine-derived stem cells on collagen synthesis in human skin fibroblasts, use was made of medium compositions containing exosomes extracted from the culture medium of human urine-derived stem cells and exosomes extracted from human adipose-derived stem cells (AD-exo), respectively. The medium compositions were prepared by adding UD-exo at a concentration of 250 μg/mL to DMEM serum-free culture medium AD-exo at a concentration of 250 μg/mL to DMEM serum-free culture medium, respectively. DMEM serum-free medium was used as a negative control. Human skin fibroblasts were seeded at a density of 3×10⁵ cells/well in 6-well plates, and incubated in culture medium (DMEM supplemented with 10% fetal bovine serum and 1% penicillin/streptomycin) for 24 hours. After the cells adhered well to the wells, the cells were washed with phosphate-buffered saline and treated with the medium compositions containing exosomes for 48 hours. To check the gene expression levels of collagen, human skin fibroblasts and culture medium from each well were collected separately. Skin fibroblasts were harvested by centrifuging at 25° C. and 2000 rpm for 5 minutes, and the culture medium was obtained by centrifuging at 25° C. and 3000 rpm for 10 minutes. The total RNA obtained from the collected skin fibroblasts was used to synthesize cDNA. Then, real-time PCR was performed using the primers shown in Table 4 to measure the change in mRNA of the collagen gene. The supernatant obtained after centrifuging the collected culture medium was used to extract and quantify soluble collagen. The GAPDH gene was used as a reference standard gene for quantifying the collagen gene.

TABLE 4

| | Primer sequence | |
|---|---|---|
| | Forward primer | Reverse primer |
| Gene | (5′→3′) | (5′→3′) |
| GAPDH | AGCCACATCGCTCAGACAC | GCCCAATACGACCAAATCC |
| Collagen | GAGTGGTGATCGTGGTGAGAC | CTTTATGCCTCTGTCGCCCTG |
| (Col1A1 type) | TGGT | TTCG |

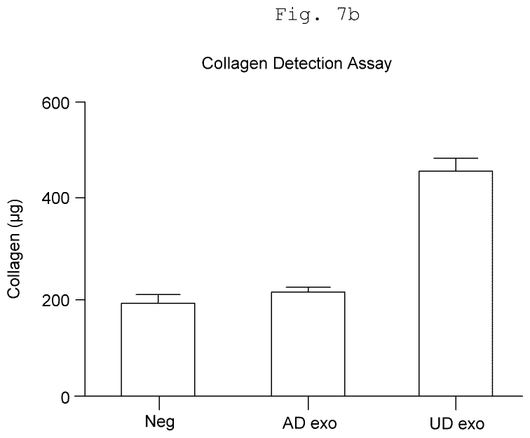

Both the human skin fibroblasts treated with culture medium containing AD-exo and UD-exo exhibited increased mRNA expression levels of collagen, compared to the negative control, with a more significant increase in the UD-exo-containing culture medium than in the AD-exo-containing culture medium (FIG. 7a).

Furthermore, a Sirius Red Total Collagen Detection Assay Kit (Cat #. 9062P, Woodinville, USA) was used to quantify the collagen in the culture media of human skin fibroblasts treated with the exosomes. The obtained supernatant was treated with a 0.05M acetic acid buffer and maintained at 4° C. for more than 12 hours. Subsequently, the provided collagen adsorbing dye (sirius red solution) was added to the supernatant to which the 0.05M acetic acid buffer was added, and centrifuged at 12,000 rpm for 10 minutes to concentrate the collagen. Afterwards, the supernatant was removed to eliminate unadsorbed dye, and the pellet was washed with a washing buffer. Then, it was centrifuged for another 10 minutes to further concentrate the collagen. Thereafter, the dye adsorbed to the collagen was dissolved by treatment with an alkali reagent, and the absorbance was measured at a wavelength of 530 nm. The amount of soluble collagen in the well with added UD-exo, AD-exo, and negative control material was calculated by substituting the absorbance into the formula of the calibration curve.

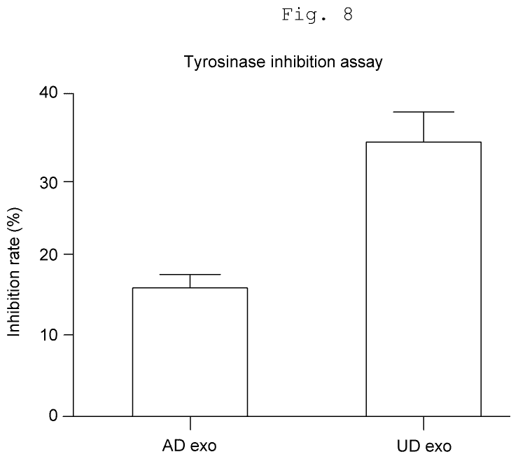

As a result, the UD-exo-administered group increased in the synthesis rate of soluble collagen, compared to the negative control group (190 μg). In particular, the collagen synthesis amount in the group given 250 μg/mL of UD-exo was 457 μg, which significantly increased compared to the same amount of AD-exo (211 μg) (FIG. 8b).

Therefore, the exosomes extracted from human adipose-derived stem cells were proven to promote collagen synthesis in skin fibroblasts.

### Example 7. Inhibitory Effect of Exosomes Extracted from Human Urine-Derived Stem Cells Using Melanocytes on Tyrosinase Activity

An examination was made to investigate the inhibitory effect of exosomes extracted from human urine-derived stem cells on tyrosinase activity. In this regard, a tyrosinase inhibition assay (abcam, cat #204715) was conducted using exosomes extracted from human urine-derived stem cell culture medium (UD-exo) and exosomes extracted from human adipose-derived stem cell culture medium (AD-exo). As tyrosinase inhibition samples, exosomes extracted from human urine-derived stem cells and human adipose-derived stem cells were each used at a concentration of 250 μg/mL. Then, the tyrosinase and tyrosinase substrate were reacted, followed by adding each exosome sample thereto. The activity of tyrosinase was compared using a microplate reader at 3-minute intervals from 30 to 60 minutes, based on the negative control group in which the enzyme-substrate reaction was conducted with no exosomes.

As a result, tyrosinase activity was decreased 34% by the group treated with exosomes extracted from human urine-derived stem cells and 16% by the group treated with exosomes extracted from human adipose-derived stem cells, demonstrating that exosomes extracted from human urine-derived stem cells have superior inhibitory activity against tyrosinase, compared to exosomes extracted from human adipose-derived stem cells.

1. A cosmetic composition, comprising exosomes derived from urine-derived stem cells as an active ingredient for skin regeneration, skin whitening, antioxidation, or wound healing.

2. The cosmetic composition of claim 1, wherein the urine-derived stem cells are cultured in a medium composition comprising: a basic medium containing Dulbecco's Modified Eagle Medium (DMEM) and Ham's F12 at a 3:1 ratio; 2.5% or less of fetal bovine serum; and an additive, wherein the additive is at least one selected from a group consisting of bovine serum albumin, a lipid mixture (chemically defined lipids), FGF23 (fibroblast growth factor 23), T3 (triiodothyronine), and calcifediol.

3. The cosmetic composition of claim 2, wherein the medium composition for extracting exosomes from the urine-derived stem cells is serum-free and antibiotic-free.

4. The cosmetic composition of claim 1, wherein the urine-derived stem cells are autologous, allogeneic, or xenogeneic.

5. The cosmetic composition of claim 1, wherein the wound comprises cuts, incisions (e.g., surgical incisions), abrasions, thermal injuries or lacerations, fractures, contusions, burns, or amputations.

6. The cosmetic composition of claim 1, wherein the cosmetic composition is formulated into skin lotions, skin softeners, toners, astringents, lotions, milk lotions, moisturizing lotions, nutritional lotions, massage creams, nutritional creams, eye creams, moisturizing creams, hand creams, essences, nutritional essences, packs, cleansing foams, cleansing waters, cleansing lotions, cleansing creams, body lotions, body cleansers, soaps, and powders.

7. A pharmaceutical composition, comprising exosomes derived from urine-derived stem cells as an active ingredient for skin regeneration, skin whitening, antioxidation, or wound healing.

8. The pharmaceutical composition of claim 7, wherein the urine-derived stem cells are cultured in a medium composition comprising: a basic medium containing Dulbecco's Modified Eagle Medium (DMEM) and Ham's F12 at a 3:1 ratio; 2.5% or less of fetal bovine serum; and an additive, wherein the additive is at least one selected from a group consisting of bovine serum albumin, a lipid mixture (chemically defined lipids), FGF23 (fibroblast growth factor 23), T3 (triiodothyronine), and calcifediol.

9. The pharmaceutical composition of claim 8, wherein the medium composition for extracting exosomes from the urine-derived stem cells is serum-free and antibiotic-free.

10. The pharmaceutical composition of claim 7, wherein the urine-derived stem cells are autologous, allogeneic, or xenogeneic.

11. The pharmaceutical composition of claim 7, wherein the wound comprises cuts, incisions (e.g., surgical incisions), abrasions, thermal injuries or lacerations, fractures, contusions, burns, or amputations.

12. The pharmaceutical composition of claim 7, wherein the pharmaceutical composition is a topical skin formulation selected from creams, gels, ointments, skin emulsions, skin suspensions, transdermal patches, mask packs, medicated bandages, lotions, or a combination thereof.

13. A method for producing exosomes derived from urine-derived stem cells for use in skin regeneration, skin whitening, antioxidation, or wound healing, the method comprising the steps of:
culturing urine-derived stem cells in a medium composition including a basic medium containing Dulbecco's Modified Eagle Medium (DMEM) and Ham's F-12 at a 3:1 ratio, 2.5% or less of fetal bovine serum, and an additive, and
culturing the cultured urine-derived stem cells in a serum-free and antibiotic-free medium composition to extract exosomes.

14. The method of claim 13, wherein the additive comprises bovine serum a albumin, lipid mixture (chemically defined lipids), FGF23 (fibroblast growth factor 23), T3 (triiodothyronine), and calcifediol.

15. The method of claim 13, wherein the serum-free and antibiotic-free culture solution comprises a basic medium containing Dulbecco's Modified Eagle Medium (DMEM) and Ham's F-12 at a 3:1 ratio, fibroblast growth factor 2 (FGF2), hydrocortisone, T3 (triiodothyronine), calcitriol, insulin-transferrin-selenium-ethanolamine, L-ascorbic acid 2-phosphate, and retinoic acid.
